(19) United States
(12) Patent Application Publication
(10) Pub. No.: US 2017/0173306 A1
(43) Pub. Date: Jun. 22, 2017

(54) SPITTABLE NEEDLE

(71) Applicants: Georgia Tech Research Corporation, Atlanta, GA (US); Emory University, Atlanta, GA (US)

(72) Inventors: Gautam Kumar, Atlanta, GA (US); Sivakkumar Arjunon, Atlanta, GA (US); Yegor Podgorsky, Lilburn, GA (US); Ajit Yoganathan, Tucker, GA (US)

(21) Appl. No.: 15/128,541

(22) PCT Filed: Mar. 24, 2015
(86) PCT No.: PCT/US15/22207

§ 371 Date: Sep. 23, 2016

Related U.S. Application Data
(60) Provisional application No. 61/969,287, filed on Mar. 24, 2014.

(51) Int. Cl. A61M 25/06; A61M 25/00; A61B 10/02; A61M 25/09

(52) U.S. Cl. CPC A61M 25/0668; A61M 25/065; A61M 25/09; A61M 25/0097; A61B 10/0233

(57) ABSTRACT

Splittable needles for use in catheterizations are described. The splittable needles are formed of equal or unequal longitudinal parts that precisely align and lock for secure access to subject's vessels. The splittable needles are provided with a clamp, which has radially protruding handles and alignment and locking mechanisms. The splittable needles precisely split and separate into respective individual parts following pressing of clamp handles together.

### CROSS REFERENCE TO RELATED APPLICATIONS

This application claims benefit of U.S. Provisional Application No. 61/969,287, filed Mar. 24, 2014, which is hereby incorporated herein by reference in its entirety.

### FIELD OF INVENTION

The present invention is generally related to splittable needles for use as medical devices aiding cardiac catheterization and peripheral and coronary intervention.

### BACKGROUND OF THE INVENTION

Splittable needles are used for aiding catheterization to the heart (cardiac catheterization), bladder (urinary catheterization), or other lumens of the body. During cardiac catheterization, a long, thin, flexible catheter is put into a blood vessel in the arm, upper thigh, or neck of a subject and threaded to the heart. Through the catheter, diagnostic tests and treatments on the heart can be performed.

Catheters are usually placed using introducer needles, catheter introducers and guidewires. Catheter introducers are typically used in conjunction with peripherally inserted central catheters (PICC), or other relatively long and flexible medical devices, to facilitate insertion and placement of the catheter or other medical device into the patient's vasculature. Current catheter introducers include a splittable cannula and a hub with a pair of wings fixed to the proximal end of the cannula. In addition, such a catheter introducer includes an introducer needle located inside the splittable cannula with its sharp distal tip extending distally of the distal end of the splittable cannula and with its needle hub extending proximal of the wings and hub on the cannula.

When using a typical catheter introducer, the clinician grasps the needle hub so the needle bevel is facing away from the patient's skin and proceeds to insert the distal portion of the needle at the desired site in the patient's skin. The clinician continues to advance the device until venous or arterial access has been confirmed. This confirmation is usually done visually when the clinician sees blood entering a flashback chamber formed in the needle hub at the proximal end of the needle or out of the open end of the needle. After vessel puncture has been confirmed, the clinician advances the cannula distally into the patient's vessel and the needle is withdrawn. With the catheter introducer properly placed, the clinician can then insert the PICC, or other relatively long, thin and flexible medical device, into the proximal opening of the cannula and continue to advance the catheter through the catheter introducer until the catheter is properly placed in the patient's vasculature.

Alternatively, the introducer needle can be first placed into the patient's vasculature without the catheter introducer. A guidewire is then inserted through the introducer needle into the patient's vasculature. The introducer needle is then retracted leaving the guidewire in place to provide a track or guide for the catheter introducer, and dilator if used, to follow into the patient's vasculature. A catheter is then inserted into the catheter introducer over the guidewire. This greatly facilitates the placement of a line into a patient's vasculature. After placement of the PICC, the clinician grasps the wings and pulls them apart to split the splittable introducer. In this way, the splittable introducer can be removed from the patient over any hub located on the proximal end of the PICC.

Examples of such devices are described in U.S. Pat. No. 4,957,488, U.S. Pat. No. 6,027,480, U.S. Pat. No. 8,758,302, and U.S. Pat. No. 8,974,411; in U.S. Publication Nos. US 2007/0135768, US 2009/0187147, US 2012/0157854, and US 2013/0217989; and in a U.K. Publication No. GB 2 278 060.

However, some of these devices are no longer available for clinical use and have been discontinued due to malfunctioning. For example, following placement of a PICC with a break-away splittable needle introducer, the needle did not split along its entire length. The two parts of the splittable needle remained connected at the junction of the needle and the hub (Device: Splittable Needle Introducer. MedSun Newsletter, 61, June 2011). Another splittable needle introducer for PICC and Midline catheters was reported to malfunction on at least three separate occasions. The splittable needle introducer did not split following placement of the PICC, causing removal of the entire line (http://www.patientsville.com/devices/splittable-needle-introducer.htm; accessed Mar. 16, 2015).

These malfunctions indicate that there remains a need for splittable needles with efficient alignment, locking and splitting mechanisms.

Therefore, it is an object of the present invention to provide splittable needles with parts that efficiently split along the entire length of the needle for safe removal of the splittable needle.

It is a further object of the present invention to provide splittable needles with parts that align and lock for efficient catheter delivery.

### SUMMARY OF THE INVENTION

Described herein are splittable needle assemblies having a hollow shaft and opposed distal ends and proximal ends, the distal ends having a tip for insertion into a body and the proximal ends having a clamp and a locking hub. The hollow shafts of the splittable needle assemblies split into at least two elements. The at least two elements are joined together by the clamp.

In some embodiments, the clamp is positioned at the proximal end of the hollow shaft before the locking hub and has an alignment element for aligning the at least two elements and tightly joining together to form the hollow shaft of the splittable needle. In some embodiments, the clamp includes at least two handles and a securing region, wherein the at least two handles are radially protruding from the securing region, and are flexibly joined. In other embodiments, the clamp is a spring-loaded clamp.

In preferred embodiments, the alignment element is located at the securing region of the clamp. The alignment element is formed of matingly-fitted elements comprising protrusions, recessions, cuts, and stepped ridges.

In some embodiments, the at least two elements of the splittable needle assembly separate when the clamp handles are brought closer together. In other embodiments, the at least two elements separate when introduced into a body lumen.

In some embodiments, the at least two elements are magnetically joined together to form the hollow shaft of the splittable needle. In other embodiments, the at least two elements are coated by a polymer-mixture coating and joined together to form the hollow shaft of the splittable needle. In yet other embodiments, the at least two elements are joined together with a biodegradable adhesive to form the hollow shaft of the splittable needle.

Generally, the splittable needles described herein are assembled to form needles with sizes ranging from 7 Gauge (g) to 33 g.

Generally, the locking hub of the splittable needle assemblies described herein is configured for locking with additional devices comprising syringes, delivery devices, valves, filters, pumps and pouches. In some embodiments, the splittable needle assemblies described herein have a locking hub with a locking alignment element. Typically, the locking alignment element is formed of matingly-fitted elements comprising protrusions, recessions, cuts, and stepped ridges.

Also provided are kits containing a plurality of individually wrapped and sterilized splittable needle assemblies and instructions for use.

Also provided are methods of accessing a body lumen or tissue of a subject using the splittable needle assemblies described herein.

### BRIEF DESCRIPTION OF THE DRAWINGS

FIG. 1 is a perspective view of the splittable needle assembly 1000 showing the splittable needle 200, the clamp 100 and the locking hub 300.

FIG. 2 is a floor plan view of the splittable needle assembly 1000, showing the splittable needle 200, the clamp 100, the locking hub 300 and the symmetry of the assembly along the longitudinal axis X.

FIG. 3 is a section view of the assembly 1000 showing parts of the splittable needle 200, the clamp 100, the locking hub 300 and the opening 400 for funneling a guidewire.

FIG. 4A is a front elevation view of the splittable needle 200 assembled with the clamp 100 along the longitudinal plane A. FIG. 4B is an enlarged view of an alignment mechanism the clamp 100.

FIG. 5A is a front elevation view of the clamp 500 showing another embodiment of the alignment mechanism. FIG. 5B is an enlarged view of the alignment mechanism of the clamp 500.

FIG. 6A is a front elevation view of the clamp 600 showing another embodiment of the alignment mechanism. FIG. 6B is an enlarged view of the alignment mechanism of the clamp 600.

FIG. 7A is a perspective view of the clamp 700, showing another embodiment of the flexing region. FIG. 7B is another perspective view of the clamp 700, showing another embodiment of the flexing region.

FIG. 8A is a perspective view of a splittable needle in split form and without a clamp, showing the locking hub 800 according to another embodiment of the splittable needle. FIG. 8B is a front elevation view of the locking hub 800 in split form. FIG. 8C is a perspective view of a splittable needle in closed form and without a clamp, showing the locking hub 800 according to another embodiment of the splittable needle. FIG. 8D is a front elevation view of the locking hub 800 in closed form.

### DETAILED DESCRIPTION OF THE INVENTION

### I. Definitions

As used herein, the term “splittable needle” refers to a needle in a shape of a tube with a hollow shaft that is splittable to at least two equal or unequal longitudinal elements.

As used herein, the term “splittable needle assembly” refers to a device containing individual parts assembled together. The individual parts include two or more longitudinal elements of the splittable needle and clamp parts.

As used herein, the term “clamp parts” refer to the elements of a device, that when assembled, form a clamp.

As used herein, “longitudinal axis X” refers to a longitudinal axis that runs from the front end F to the rear end R of the splittable needle and passes through the bore of the needle.

As used herein, “longitudinal plane A” refers to a plane that passes through the longitudinal axis X of the splittable needle, dividing the needle into symmetric halves.

As used herein, the term “front end F” and the term “distal end” refer to the distal tip of a splittable needle that is located away from the locking hub, and that pierces and enters subject's tissue.

As used herein, the term “rear end R” and the term “proximal end” refer to the proximal end of a splittable needle located opposite to the front end F, and harboring the locking hub.

As used herein, the term “alignment” refers to a position of one part of the splittable needle relative to the other. As such, the term “alignment mechanism” refers to a mechanism that places one part of the splittable needle precisely aligned with the other part of the needle when the needle is assembled.

As used herein, the term “locking” refers to the action of locking one part of the splittable needle into the other. As such, the term “locking mechanism” refers to a mechanism that achieves locking of one part of the splittable needle into the other without disturbing the alignment of the assembled splittable needle.

As used herein, the term “splitting” refers to the action of separating, splitting, pulling, tearing, or peeling the individual longitudinal parts of the splittable needle from each other back into the respective individual longitudinal parts.

As used herein, the term “matingly” refers to a manner of joining at least two elements, wherein one element is configured to tightly fit within the other element.

### II. Splittable Needles

### A. Splittable Needle Assembly

Generally, the splittable needle assembly described herein has splittable needle, a locking hub and a clamp.

1. The Needle

The splittable needle is a needle split into equal or unequal parts along its longitudinal axis. The splittable needle has a tip region located at the front end of the needle.

a. The Needle Tip

The tip region may have any shape, including, but not limited to cone shape, dual gauge, bevel of various degrees, 90 degree cut (such as those used in percutaneous coronary intervention), domed and side hole domed. Typically, the tip region is beveled for ease of entry to a body lumen of a subject in need thereof.

b. The Needle Length

Typically, the needle has a length needed to reach a desired vessel. Generally, the needle length varies from 2 cm to 20 cm. Preferably, the needle length ranges from 5 cm to 9 cm. Most preferably, the needle length is 7 cm.

c. Needle Size

The needle has an external diameter and an internal bore diameter corresponding to a conventional needle gauge dimensions. A conventional needle gauge chart is presented in Table 1. Generally, the needle size is any gauge (g) that can accommodate the guidewire and the catheter assembly. Typically, the needle size ranges from 7 g to 33 g. In preferred embodiments, the range for the external diameter is from 0.75 mm to 1.15 mm, and for the internal diameter is from 0.45 to 0.75 mm, corresponding to 18 g or 21 g hypodermic needles. The internal diameter may be wider than the specified range if the needle is thin walled.

TABLE 1
A list of conventional needle gauge dimensions.

| | | | | Nominal Inner Diameter | | | | | |
|---|---|---|---|---|---|---|---|---|---|
| | Nominal Outer Diameter | | | | | tol. | Nominal Wall Thickne | | |
| Needle | | | tol. inches | | | inches | | | tol. inches |
| Gauge | inches | mm | (mm) | inches | mm | (mm) | inches | mm | (mm) |
| 7 | 0.180 | 4.572 | ±0.001 | 0.150 | 3.810 | ±0.003 | 0.015 | 0.381 | ±0.001 |
| | | | (±0.025) | | | (±0.076) | | | (±0.025) |
| 8 | 0.165 | 4.191 | ±0.001 | 0.135 | 3.429 | ±0.003 | 0.015 | 0.381 | ±0.001 |
| | | | (±0.025) | | | (±0.076) | | | (±0.025) |
| 9 | 0.148 | 3.759 | ±0.001 | 0.118 | 2.997 | ±0.003 | 0.015 | 0.381 | ±0.001 |
| | | | (±0.025) | | | (±0.076) | | | (±0.025) |
| 10 | 0.134 | 3.404 | ±0.001 | 0.106 | 2.692 | ±0.003 | 0.014 | 0.356 | ±0.001 |
| | | | (±0.025) | | | (±0.076) | | | (±0.025) |
| 11 | 0.120 | 3.048 | ±0.001 | 0.094 | 2.388 | ±0.003 | 0.013 | 0.330 | ±0.001 |
| | | | (±0.025) | | | (±0.076) | | | (±0.025) |
| 12 | 0.109 | 2.769 | ±0.001 | 0.085 | 2.159 | ±0.003 | 0.012 | 0.305 | ±0.001 |
| | | | (±0.025) | | | (±0.076) | | | (±0.025) |
| 13 | 0.095 | 2.413 | ±0.001 | 0.071 | 1.803 | ±0.003 | 0.012 | 0.305 | ±0.001 |
| | | | (±0.025) | | | (±0.076) | | | (±0.025) |
| 14 | 0.083 | 2.108 | ±0.001 | 0.063 | 1.600 | ±0.003 | 0.01 | 0.254 | ±0.001 |
| | | | (±0.025) | | | (±0.076) | | | (±0.025) |
| 15 | 0.072 | 1.829 | ±0.0005 | 0.054 | 1.372 | ±0.0015 | 0.009 | 0.229 | ±0.0005 |
| | | | (±0.013) | | | (±0.038) | | | (±0.013) |
| 16 | 0.065 | 1.651 | ±0.0005 | 0.047 | 1.194 | ±0.0015 | 0.009 | 0.229 | ±0.0005 |
| | | | (±0.013) | | | (±0.038) | | | (±0.013) |
| 17 | 0.058 | 1.473 | ±0.0005 | 0.042 | 1.067 | ±0.0015 | 0.008 | 0.203 | ±0.0005 |
| | | | (±0.013) | | | (±0.038) | | | (±0.013) |
| 18 | 0.050 | 1.270 | ±0.0005 | 0.033 | 0.838 | ±0.0015 | 0.0085 | 0.216 | ±0.0005 |
| | | | (±0.013) | | | (±0.038) | | | (±0.013) |
| 19 | 0.042 | 1.067 | ±0.0005 | 0.027 | 0.686 | ±0.0015 | 0.0075 | 0.191 | ±0.0005 |
| | | | (±0.013) | | | (±0.038) | | | (±0.013) |
| 20 | 0.03575 | 0.9081 | ±0.00025 | 0.02375 | 0.603 | ±0.00075 | 0.006 | 0.1524 | ±0.00025 |
| | | | (±0.0064) | | | (±0.019) | | | (±0.0064) |
| 21 | 0.03225 | 0.8192 | ±0.00025 | 0.02025 | 0.514 | ±0.00075 | 0.006 | 0.1524 | ±0.00025 |
| | | | (±0.0064) | | | (±0.019) | | | (±0.0064) |
| 22 | 0.02825 | 0.7176 | ±0.00025 | 0.01625 | 0.413 | ±0.00075 | 0.006 | 0.1524 | ±0.00025 |
| | | | (±0.0064) | | | (±0.019) | | | (±0.0064) |
| 22s | 0.02825 | 0.7176 | ±0.00025 | 0.006 | 0.152 | ±0.00075 | 0.0111 | 0.2826 | ±0.00025 |
| | | | (±0.0064) | | | (±0.019) | | | (±0.0064) |
| 23 | 0.02525 | 0.6414 | ±0.00025 | 0.01325 | 0.337 | ±0.00075 | 0.006 | 0.1524 | ±0.00025 |
| | | | (±0.0064) | | | (±0.019) | | | (±0.0064) |
| 24 | 0.02225 | 0.5652 | ±0.00025 | 0.01225 | 0.311 | ±0.00075 | 0.005 | 0.1270 | ±0.00025 |
| | | | (±0.0064) | | | (±0.019) | | | (±0.0064) |
| 25 | 0.02025 | 0.5144 | ±0.00025 | 0.01025 | 0.260 | ±0.00075 | 0.005 | 0.1270 | ±0.00025 |
| | | | (±0.0064) | | | (±0.019) | | | (±0.0064) |
| 26 | 0.01825 | 0.4636 | ±0.00025 | 0.01025 | 0.260 | ±0.00075 | 0.004 | 0.1016 | ±0.00025 |
| | | | (±0.0064) | | | (±0.019) | | | (±0.0064) |
| 26s | 0.01865 | 0.4737 | ±0.00025 | 0.005 | 0.127 | ±0.00075 | 0.0068 | 0.1734 | ±0.00025 |
| | | | (±0.0064) | | | (±0.019) | | | (±0.0064) |
| 27 | 0.01625 | 0.4128 | ±0.00025 | 0.00825 | 0.210 | ±0.00075 | 0.004 | 0.1016 | ±0.00025 |
| | | | (±0.0064) | | | (±0.019) | | | (±0.0064) |
| 28 | 0.01425 | 0.3620 | ±0.00025 | 0.00725 | 0.184 | ±0.00075 | 0.0035 | 0.0889 | ±0.00025 |
| | | | (±0.0064) | | | (±0.019) | | | (±0.0064) |
| 29 | 0.01325 | 0.3366 | ±0.00025 | 0.00725 | 0.184 | ±0.00075 | 0.003 | 0.0762 | ±0.00025 |
| | | | (±0.0064) | | | (±0.019) | | | (±0.0064) |
| 30 | 0.01225 | 0.3112 | ±0.00025 | 0.00625 | 0.159 | ±0.00075 | 0.003 | 0.0762 | ±0.00025 |
| | | | (±0.0064) | | | (±0.019) | | | (±0.0064) |
| 31 | 0.01025 | 0.2604 | ±0.00025 | 0.00525 | 0.133 | ±0.00075 | 0.0025 | 0.0635 | ±0.00025 |
| | | | (±0.0064) | | | (±0.019) | | | (±0.0064) |
| 32 | 0.00925 | 0.2350 | ±0.00025 | 0.00425 | 0.108 | ±0.00075 | 0.0025 | 0.0635 | ±0.00025 |
| | | | (±0.0064) | | | (±0.019) | | | (±0.0064) |
| 33 | 0.00825 | 0.2096 | ±0.00025 | 0.00425 | 0.108 | ±0.00075 | 0.002 | 0.0508 | ±0.00025 |
| | | | (±0.0064) | | | (±0.019) | | | (±0.0064) |
| 34 | 0.00725 | 0.1842 | ±0.00025 | 0.00325 | 0.0826 | ±0.00075 | 0.002 | 0.0508 | ±0.00025 |
| | | | (±0.0064) | | | (±0.019) | | | (±0.0064) |

d. Needle Materials

The needle and the individual needle parts may be formed of any biocompatible needle material with desired stealth properties and resistance to corrosion. Typically, the needle parts are formed of stainless steel and is coated with nickel to prevent corrosion.

In some embodiments, the separate parts of the splittable needle may have magnetized edges. The magnetized edges may be present along the entire circumference of the edges of the needle parts. In other embodiments, only portions of the circumference of the needle edges are magnetized. The magnetized edges help with the alignment and securement of the two needle parts relative to each other to form the splittable needle for safe and secure tissue puncture and penetration.

In embodiments with magnetized edges, ferromagnetic composites are formed using methods known in the art (Zhong et al., Magnetics Conference, INTERMAG Asia 2005. Digests of the IEEE International. doi: 10.1109/INTMAG.2005.1463871 (2005)). The magnetized needles can be manufactured through powdered metallurgy methods.

In some embodiments, the edges of the needle parts are coated with a biocompatible adhesive. The adhesive allows the needle parts to come together and be held together until splitting of the needle is required. The adhesive is designed to be of a certain strength so that force applied to separate the two needle parts overcomes the adhesive strength of the adhesive and the two needle parts are separated.

In some embodiments, the separate needle parts are formed of flexible materials, such as metal alloys, and are held together by a biocompatible coating or adhesive. Once inserted into the patient, the coating or adhesive is dissolved in blood. The needle is then split into two following retraction from the vessel. In this embodiment, the front of the needle is split due to dissolving of the coating or the adhesive, while the rear of the needle is split due to a force applied to the clamps to separate the needle. In preferred embodiments, the needle parts are held together by a polymer-mixture coating like cyanoacrylates, methacrylates, epoxy, dimethacrylate esters, silicones, urethanes, phenol-formaldehydes.

2. The Locking Hub

In some embodiments, the splittable needle has a locking hub located at the rear end of the needle. Typically, the locking hub is adapted for locking with additional devices, including, but not limited to, syringes, delivery devices, valves, filters, pumps and pouches. Generally, the locking hub is formed of locking hub parts, with each part attached to a respective needle part of the splittable needle. When assembled, the locking hub parts come together to form the locking hub, just as the needle parts come together and align, forming the splittable needle assembly. In preferred embodiments, the locking hub is threaded and forms a threaded adapter, such as a Luer Lok™ (Becton Dickinson) when the splittable needle is assembled.

In some embodiments, the locking hub has a locking neck region and locking tip region. When the splittable needle assembly is split, the locking neck region and the locking tip region separate into at least two parts.

In some embodiments, the locking neck region has an alignment mechanism.

a. Alignment Mechanism of the Locking Hub

In some embodiments the at least two parts of the locking neck region have protrusions and recesses that align and fit into each other, forming an alignment mechanism. Typically, each locking neck part has at least one protrusion and/or at least one recess. When assembled, the protrusions and recesses ensure that the locking hub parts come together and align correctly, avoiding any misalignment.

b. Cavity of the Locking Hub

In a preferred embodiment, the locking hub has a cavity that extends from the locking hub to the bore of the needle. Generally, the cavity aids placement of a catheter or a guidewire into the needle. Typically, the cavity has a narrow diameter at the front end of the locking hub that matches the bore diameter of the needle. The cavity also has a wider diameter at the rear end of the locking hub where the additional devices can be locked onto the needle. This transition from a wider diameter to narrower diameter in the cavity allows for easy threading of catheters or guidewires through the needle bore.

The locking hub is typically formed of a durable plastic or metal. In preferred embodiments, the locking hub is formed of durable plastic.

3. The Clamp

Generally, the splittable needle has at least one clamp. The clamp is typically positioned over and around the splittable needle. The clamp may occupy at least 1/10, 1/9, ⅛, 1/7, ⅙, ⅕, ¼ or ⅓ of the length of the splittable needle. In preferred embodiments, the clamp occupies ¼ of the length of the splittable needle. In a preferred embodiment, the clamp allows adequate needle length to reach a desired vessel.

In some embodiments, the clamp has a flexing region and an adjacent securing region. In other embodiments, the flexing region and the securing region are one and the same region.

The clamp can be made of any material with desirable flexing properties. The clamp may be metallic, hard or soft plastic, or a combination thereof. In preferred embodiments, the clamp is formed of hard plastic like polyethylene, polytetraflouroethylene, polyvinyl chloride, polypropylene, polycarbonate, polylactide.

Typically, each of the equal or unequal parts of the splittable needle is attached to at least one clamp. Either a chemical adhesive, or a technique such as ultrasonic welding, can be used to unite the needle components to the clamp. If a chemical adhesive or a bonding agent is used, the agent is preferably biocompatible.

a. The Flexing Region

Typically, the flexing region is formed of at least two clamp handles projecting radially from the needle and placed opposite to each other. In some embodiments, the clamp handles are joined together by a flexing part or a bridge.

i. Clamp Parts

Typically, the clamp has at least two clamp handles. Preferably, the two handles of the clamp are symmetrically positioned along the longitudinal plane A of the splittable needle assembly. In some embodiments, the at least two clamp handles are shaped as elongated protruding members radially extending outwards and running along the length of the clamp and along the longitudinal plane A.

Generally, the clamp handles are positioned opposite to each other and in any direction that allows for easy gripping and flexing of the clamp. In some embodiments, the clamp handles project radially in a V shape, where they approach each other at the point of attachment to the needle. In other embodiments, the clamp handles are parallel to each other. In preferred embodiments, the clamp handles are positioned in a V shape. The shape of the clamp handles is configured for easy gripping of the clamp with fingers during insertion. In some embodiments, the shape of the clamp handles is generally flat-cuboid. In other embodiments, the handles are shaped as wings, waves, or ladles. In some embodiments, the handles have imprinted indents for finger tips to allow for easy gripping.

ii. Flexing Part

In some embodiments, the flexing part is a flexible bridge positioned anywhere in between the two clamp handles and joining the two clamp handles. Typically, the flexing part joins the middle section of one of the clamp handles with the middle section of the other clamp handle along the longitudinal plane of the splittable needle assembly. In these embodiments, the flexing part has one point of attachment to one clamp handle and another point of attachment to the other clamp handle. In preferred embodiments, each of the clamp handles can swivel about the axis of its respective point of attachment. Therefore, each of the at least two clamp handles can swivel independently about the axis of its respective point of attachment to the flexing part.

Optionally, the flexing part is configured to flex at about its center. In this embodiment, each of the clamp parts can swivel about the axis of its respective point of attachment to the flexing part, and the flexing part in turn can flex along the longitudinal plane A crossing the center of the flexing part. This embodiment allows for greater degree of motion of the clamp parts relative to each other.

In preferred embodiments, each of the clamp parts can swivel about the axis of its respective point of attachment to the flexing part, and the flexing part does not flex about its center. In preferred embodiments, the clamp parts are positioned in a V shape, and approach each other to form the securing region of the clamp.

b. The Securing Region

Generally, the securing region of a clamp has needle attachment regions and regions with alignment and locking mechanisms. Typically, the needle attachment regions are in close proximity to the regions of alignment and locking mechanisms.

In some embodiments, the securing region of a clamp is divided along the longitudinal plane A of the splittable needle assembly into two unequal parts: a protruding part and a recess part. In other embodiments, the securing region is divided along the longitudinal plane of the splittable needle assembly into equal parts. In preferred embodiments, the securing region is divided into a protruding part and a recess part, which are continuations of the symmetric clamp handles of the flexing region. The protruding part fits into, and optionally, locks into, the recess part. Together, the protruding part and the recess part are the structures forming the alignment mechanism and the locking mechanism of the securing region.

i. Needle Attachment Regions

Generally, each of the two parts of the securing region has a needle attachment region. Typically, the needle attachment region is located at about the center of each of the parts. However, in some embodiments, the needle attachment region may be located off-center, or close to outer edges of each of the parts of the securing region. In preferred embodiments, the needle attachment region is located at about the center of each of the parts of the securing region.

Generally, the needle attachment region can have any shape that matches and mates with the outer surface of the needle part of the splittable needle. Typically, the needle attachment region is concave. In preferred embodiments, the concave needle attachment region has an outer surface that mates with the convex outer surface of the needle part of the splittable needle. In preferred embodiments, each of the two needle parts of the splittable needle mates with the concave needle attachment regions of each of the two parts of the securing regions.

At the needle attachment regions, the radius of the clamp securing region is less than the radius of a designated needle, to allow the needle walls to meet.

The parts of the needle can be attached to the parts of the securing region of the clamp using any suitable means. In some embodiments, the attachment is achieved through chemical means, such as biocompatible adhesives or glues. In other embodiments, the attachment is achieved through physical bonding, such as ultrasonic welding. The selection of suitable means of attachment is based on the nature of materials forming the needle parts and the clamp parts.

ii. Alignment Mechanism

In some embodiments, the protruding part of the securing region has protrusions and edges running on its surface along the length of the clamp and along the longitudinal plane A. Typically, the recess part of the securing region has recesses and notches running its surface along the length of the clamp along the longitudinal plane A. Any number of protrusions, edges, recesses and notches can be present. In a preferred embodiment, the protruding part has one protrusion and no edge, and the recess part has at least one recess and one notch. During assembly, the protrusions of one part of the splittable needle align with and fit into the recesses of the other part of the splittable needle, providing for precise alignment. Therefore, in preferred embodiments, the protrusions and recesses allow one part of the splittable needle to precisely align with the other part of the needle along the entire needle length, eliminating potential misalignments that may lead to tissue injury, or leakage of fluids into or out of the needle.

In some embodiments, the protruding part and the recess part run only ½, ⅓, ¼, ⅕, ⅙, 1/7, ⅛, 1/9, or 1/10 of the length of the clamp along its longitudinal plane. In preferred embodiments, the protruding part and the recess part run along the entire length of the clamp along its longitudinal plane.

iii. Locking Mechanism

In some embodiments, the protrusions may carry extending edges, and the recesses may include notches. The edges are configured to securely fit and lock into the notches. Any number of edges per protrusion and any matching number of notches per recess may be present as long as an effective locking is achieved.

During assembly, the two needle parts are brought close together so that the edges fit into and lock in the notches, activating the securing region's locking mechanism, while simultaneously aligning the protrusions with recesses, and activating the aligning mechanism of the securing region. Assembled in this way, any misalignment, as well as any independent movement of one needle part relative to another are prevented until the two parts are split apart.

iv. Additional Elements

The securing region may have additional elements, such as covers, coatings, appendages, or limbs. The additional elements may be added to enhance the securement function of the securing region, or to provide adapters that will aid with holding, handling, and maneuvering of the clamp and the splittable needle assembly.

### B. Exemplary Embodiments

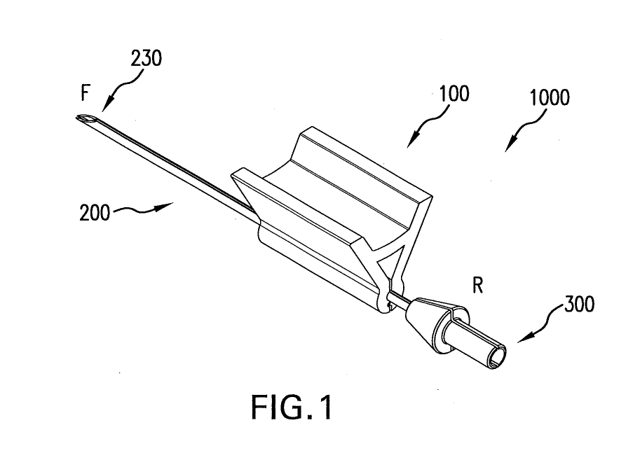

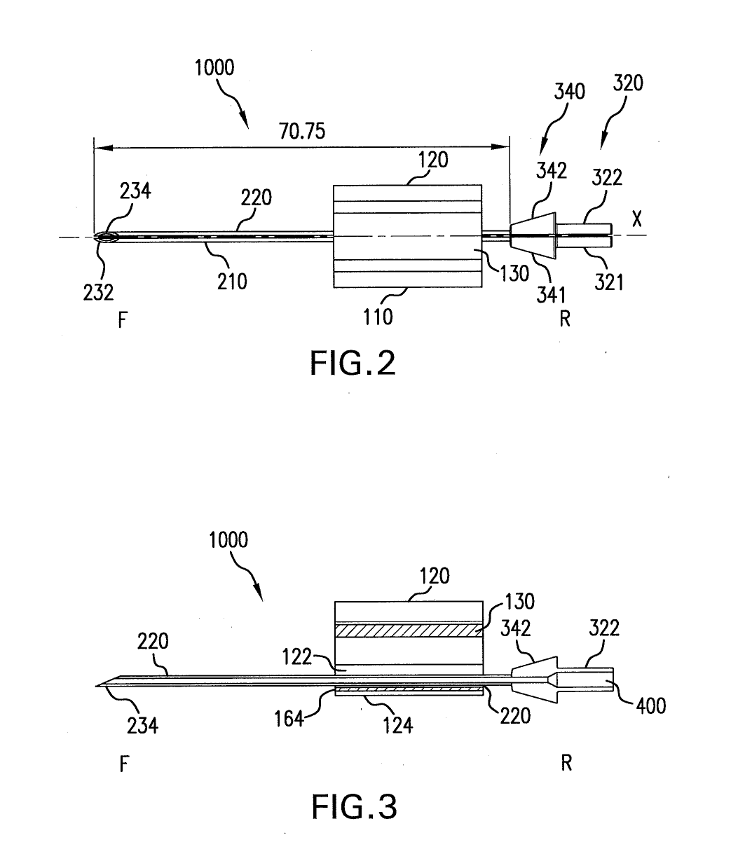

In a preferred embodiment, the splittable needle assembly is formed of a splittable needle, a clamp and a locking hub. FIGS. 1 and 2 show the splittable needle assembly 1000, with the splittable needle 200, the clamp 100 and the lock element 300. The splittable needle 200 is formed of a first needle part 210 and a second needle part 220, which are positioned opposing one another and precisely aligned along the longitudinal axis X with their tips, edges and locking hubs. The splittable needle 200 has a tip region 230 located at the front end F of the needle. The splittable needle 200 also has a locking hub 300 located at the rear end R of the needle. In one embodiment, the tip region 230 is beveled for ease of entry into a body lumen of a subject in need thereof. Therefore, the tips of first needle part 210 and the second needle part 220 are also beveled, forming the first tips 232 and the second tip 234, respectively, located at the front end F of the needle parts 210 and 220.

The locking hub 300 is positioned at the rear end R of the splittable needle assembly 1000. The locking hub 300 is formed of a locking neck region 320 and a locking tip region 340 that precedes the locking neck region 320. The locking tip region 340 has a first locking tip 341 and a second locking tip 342 that come together along the longitudinal axis X of the splittable needle assembly to form the locking tip region 340. The locking neck region 320 has a first locking neck 321 and a second locking neck 322 at the rear end R that come together along the longitudinal axis X of the splittable needle assembly 1000. The first locking neck 321 extends into a first locking tip 341, and the second locking neck 322 extends into a second locking tip 342 towards the front end F of the splittable needle 200.

Referring now to FIG. 3, the splittable needle assembly 1000 has a funnel-shaped cavity 400 within the locking hub 300. The funnel-shaped cavity 400 extends from the locking neck region 320 to the locking tip region 340 and joins with the bore of the splittable needle 200. The funnel-shaped cavity 400 has its narrow diameter end within the locking tip region 340 from where it expands in diameter and enters the locking neck region 320. The locking hub 300 with its funnel-shaped cavity 400 is adapted for receiving guidewires and catheters.

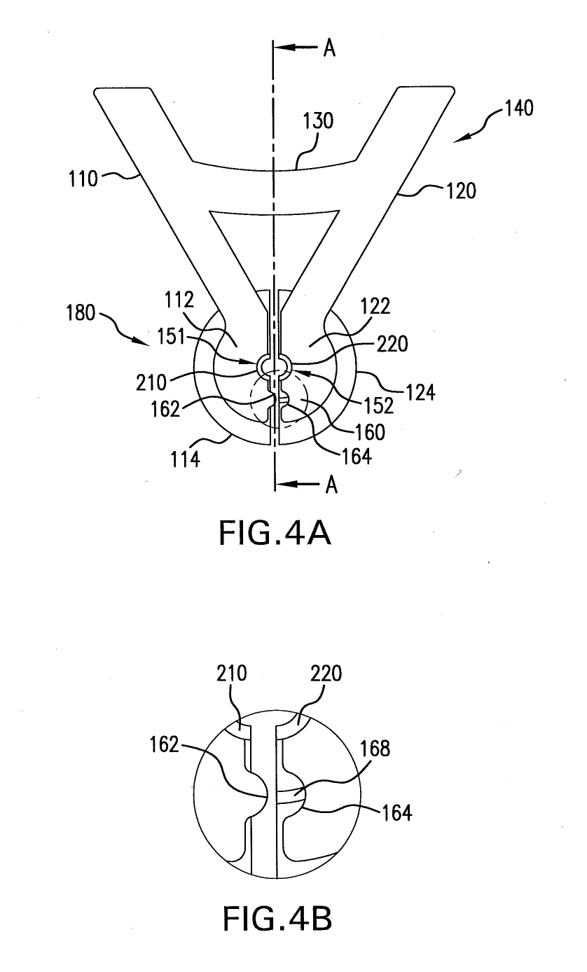

Referring now to FIG. 4A, the clamp 100 has a flexing region 140 and a securing region 180, which is attached to the splittable needle 200. The flexing region 140 is formed of a first clamp handle 110 and a second clamp handle 120 positioned opposite to each other along the longitudinal plane A of the assembled splittable needle assembly 1000. The first clamp handle 110 is joined with the second clamp handle 120 via a flexing part 130 positioned about half-way in the flexing region 140. The flexing region 140 of the clamp 100 transitions into a securing region 180, with which the clamp 100 attaches to the splittable needle 200.

The securing region 180 is formed of a first semi-circular ending 112 of the first clamp handle 110 and of a second semi-circular ending 122 of a second clamp handle 120. The first semi-circular ending 112 has a first outer surface 114 and the second semi-circular ending 122 has a second outer surface 124.

The semi-circular ending 112 has a center region for attaching to the first needle part 210—the first needle attachment region 151. The semi-circular ending 122 has a center region for attaching to the second needle part 220—the second needle attachment region 152.

At the needle attachment regions 151 and 152, the radius of the clamp securing region is less than the radius of a designated needle, to allow the edges of the needle parts to meet (see FIG. 4A). Therefore, at the needle attachment region, the needle attachment regions 151 and 152 of the clamp that hold the two needle parts together have a smaller surface area than the surface area of the outer surface of needle parts 210 and 220, so the two needle parts make tight contact.

The securing region 180 also has an alignment mechanism 160 for aligning the first needle part 210 and the second needle part 220 and locking the parts in place. In preferred embodiments, the alignment mechanism 160 is positioned in close proximity to the first needle part 210 and a second needle part 220 along the longitudinal axis A of the needle assembly 1000.

Referring now to FIGS. 4A and 4B, the alignment mechanism 160 is positioned in immediate vicinity of the first needle attachment region 151 and the second needle attachment region 152. The alignment mechanism 160 has a protruding body 162 on the end of the first semi-circular ending 112. The protruding body 162 matches in shape with and fits within a recess element 164 of the semi-circular ending 122. When assembled, the alignment mechanism 160 allows the protruding body to fit into the recess element 164 and align the first needle part 210 with the second needle part 220. The alignment mechanism 160 also secures the first needle part 210 relative to the second needle part 220. With precise manufacturing the splittable needle assembly 1000 eliminates the need of any adhesives or sealants along the length of the needle, and no leakage is expected, because the alignment mechanism 160 precisely aligns and tightly secures the first needle part 210 to the second needle part 220.

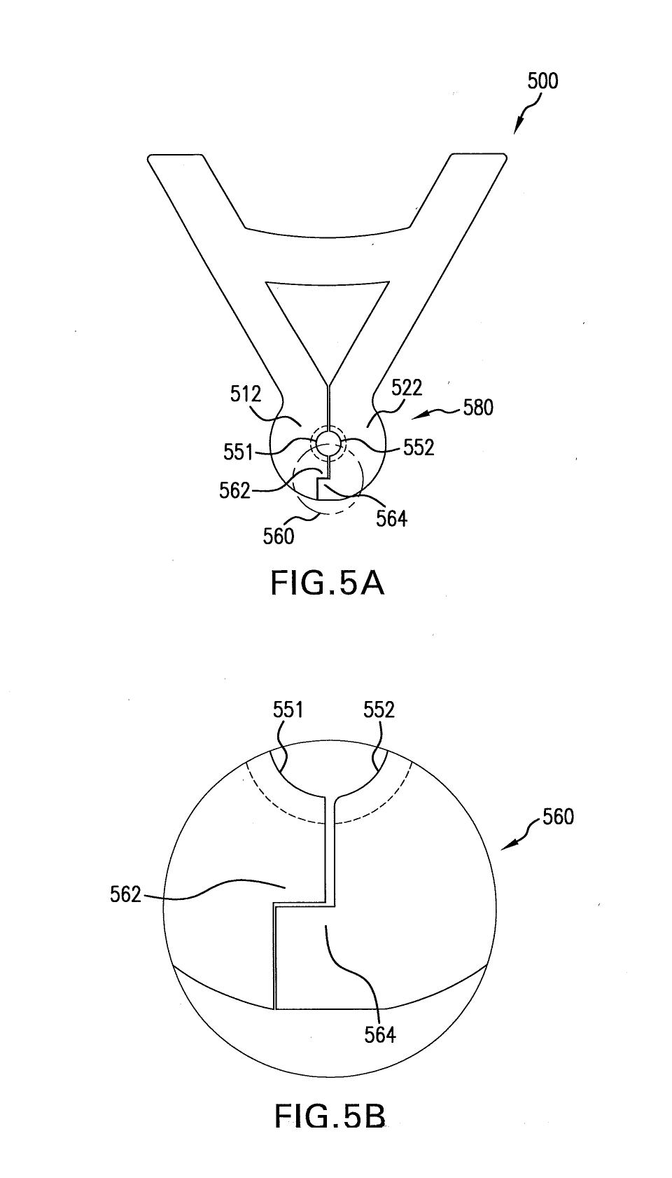

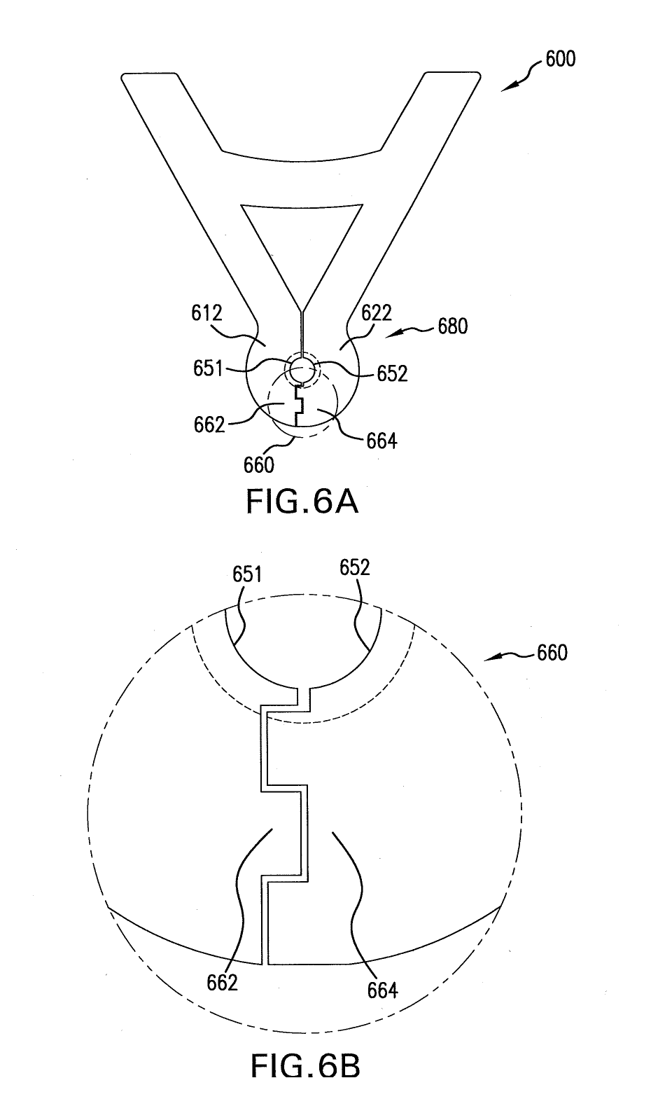

Other embodiments of the alignment mechanism are presented in FIGS. 5A-6B.

Referring now to FIGS. 5A and 5B, the clamp 500 has a securing region 580 formed a first semi-circular ending 512 and a second semi-circular ending 522. The first semi-circular ending 512 and the second semi-circular ending 522 have an alignment mechanism 560, a first needle attachment region 551 and a second needle attachment region 552. The alignment mechanism 560 is positioned in immediate vicinity of the first needle attachment region 551 and the second needle attachment region 552. The first semi-circular ending 512 has an indented cut 562. The second semi-circular end 522 has a protruding cut 564. The indented cut 562 and the protruding cut 564 run longitudinally along the length of the clamp 500. The indented cut 562 matches in shape with and fits within the protruding cut 564. When assembled, the alignment mechanism 560 allows the indented cut 562 to fit within the protruding cut 564 and align the first needle attachment region 551 with the second needle attachment region 552, as shown in FIG. 5B.

FIGS. 6A and 6B show another embodiment of the alignment mechanism. In this embodiment, the clamp 600 has a securing region 680 formed a first semi-circular ending 612 and a second semi-circular ending 622. The first semi-circular ending 612 and the second semi-circular ending 622 have an alignment mechanism 660, a first needle attachment region 651 and a second needle attachment region 652. The alignment mechanism 660 is positioned in immediate vicinity of the first needle attachment region 651 and the second needle attachment region 652. The first semi-circular ending 612 has stepped ridges 662. The second semi-circular end 622 has stepped ridges 664 that mate with the stepped ridges 662. The stepped ridges 662 664 run longitudinally along the length of the clamp 600. When assembled, the alignment mechanism 660 allows the stepped ridges 662 to align and fit within the stepped ridges 664 and align the first needle attachment region 651 with the second needle attachment region 652, as shown in FIG. 6B.

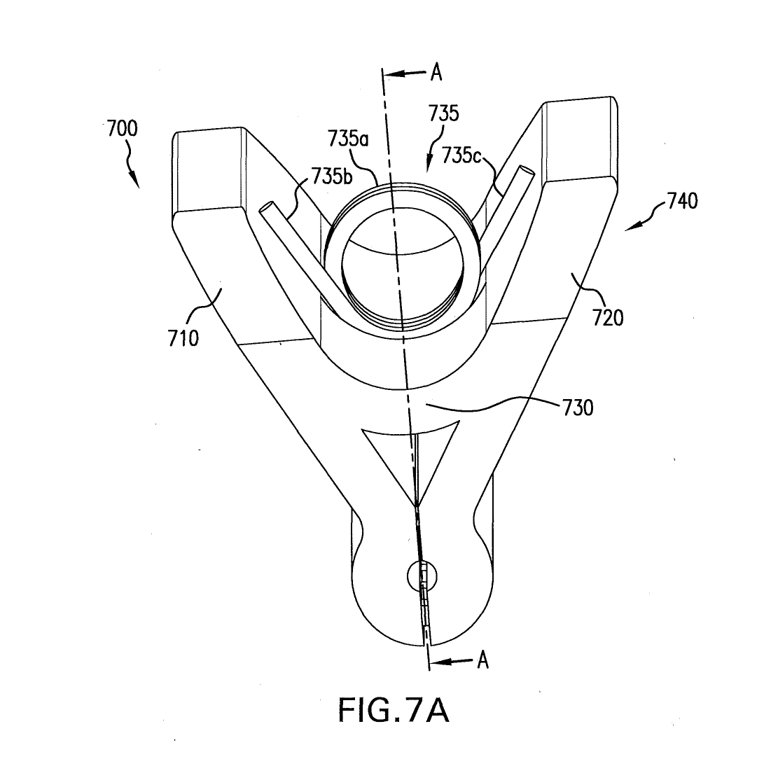

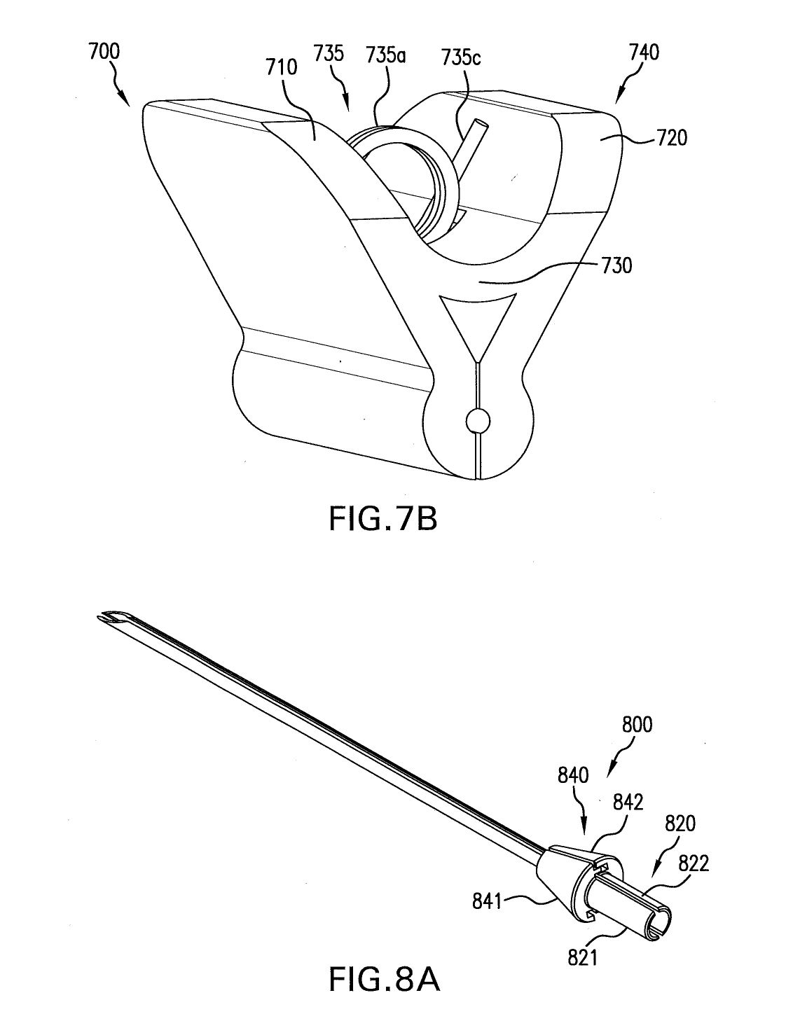

FIGS. 7A-7B present another embodiment of the flexing region of the clamp. In this embodiment, the clamp 700 has a spring-loaded flexing region 740. The flexing region 740 is formed of a first clamp handle 710 and a second clamp handle 720 positioned opposite to each other along the longitudinal plane A of the clamp. The first clamp handle 710 is joined with the second clamp handle 720 via a flexing part 730 positioned about half-way in the flexing region 740. The flexing part 730 is linked to a coiled spring 735. The coiled spring 735 has a coiled region 735a two longitudinal ends, 735b and 735c. The longitudinal end 735b is embedded in the first clamp handle 710, while the longitudinal end 735c is embedded in the second clamp handle 720. The coiled region 735a of the coiled spring 735 is partially embedded in the flexing part 730. In this embodiment, the coiled spring 735 provides the locking/clamping force, instead of relying on the natural flexing property of the flexing part 130 in the embodiment presented in FIG. 4A.

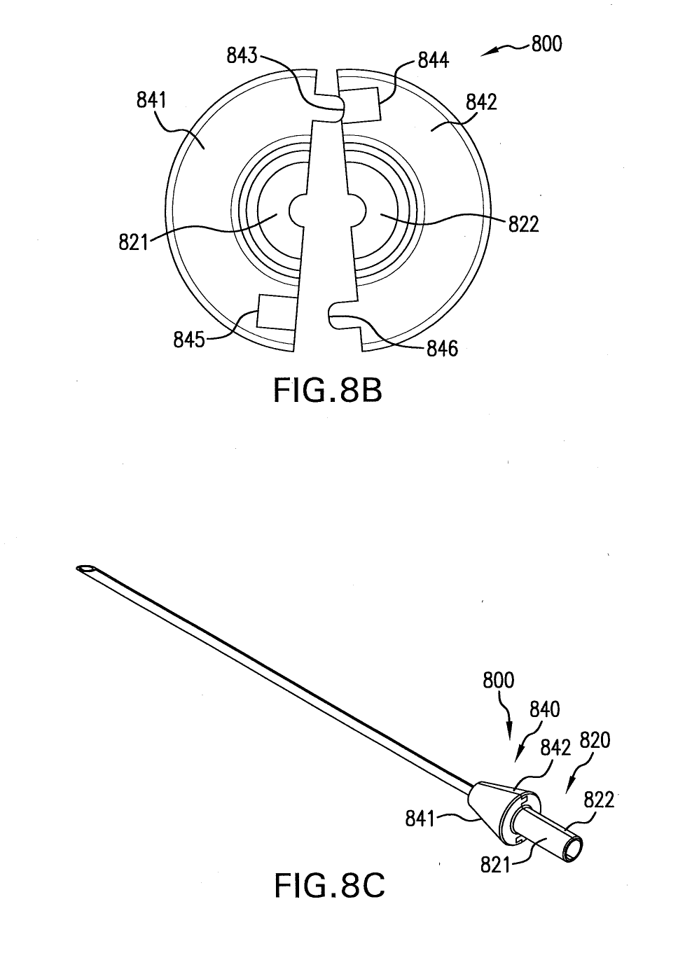

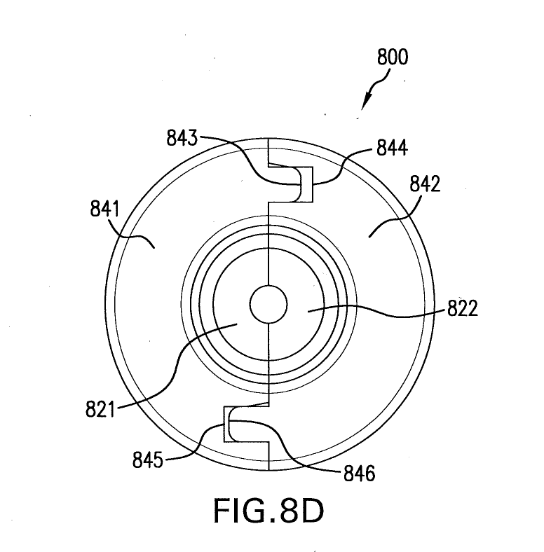

FIGS. 8A-8D present another embodiment of the locking hub for the splittable needle assembly. In this embodiment, the locking hub 800 is formed of locking neck region 820 and a locking tip region 840 that precedes the locking neck region 820. The locking tip region 840 splits into a first locking tip 841 and a second locking tip 842. The first locking tip 841 and the second locking tip 842 have a narrow front end a wide base at the rear. The first locking tip 841 has a locking protrusion 843 and a locking recess 845 at its base. The second locking tip 842 has a locking protrusion 846 and a locking recess 844 at its base. Collectively, the protrusions 843 and 846, and the recesses 844 and 845, form the alignment mechanism of the locking hub. During assembly of the splittable needle, the alignment mechanism ensures that the locking hub is aligned and securely locked. This is achieved by fitting the locking protrusion 843 of the first locking tip 841 into the locking recess 844 of the second locking tip 842, and similarly, by fitting the locking protrusion 846 of the second locking tip 842 into the locking recess 845 of the first locking tip 841.

A list of referenced numbers with corresponding labels is presented in Table 2.

TABLE 2
A list of referenced numbers and their labels.

| Label | Number |
|---|---|
| Clamp | 100 |
| First Clamp Handle | 110 |
| First Semi-Circular Ending | 112 |
| First Outer Surface | 114 |
| Second Clamp Handle | 120 |
| Second Semi-Circular Ending | 122 |
| Second Outer Surface | 124 |
| Flexing Part | 130 |
| Flexing Region | 140 |
| First Needle Attachment Region | 151 |
| Second Needle Attachment Region | 152 |
| Alignment Mechanism | 160 |
| Protruding Body | 162 |
| Recess Element | 164 |
| Locking Notch | 168 |
| Securing Region | 180 |
| Splittable Needle | 200 |
| First Needle Part | 210 |
| Second Needle Part | 220 |
| Tip Region | 230 |
| First Tip | 232 |
| Second Tip | 234 |
| Locking hub | 300 |
| Locking Neck Region | 320 |
| First Locking Neck | 321 |
| Second Locking Neck | 322 |
| Locking Tip Region | 340 |
| First Locking Tip | 341 |
| Second Locking Tip | 342 |
| Funnel Region | 400 |
| Clamp | 500 |
| Securing Region | 580 |
| First Semi-Circular Ending | 512 |
| Second Semi-Circular Ending | 522 |
| Alignment Mechanism | 560 |
| First Needle Attachment Region | 551 |
| Second Needle Attachment Region | 552 |
| Indented Cut | 562 |
| Protruding Cut | 564 |
| Clamp | 600 |
| Securing Region | 680 |
| First Semi-Circular Ending | 612 |
| Second Semi-Circular Ending | 622 |
| Alignment Mechanism | 660 |
| First Needle Attachment Region | 651 |
| Second Needle Attachment Region | 652 |
| Stepped Ridges | 662 |
| Stepped Ridges | 664 |
| Clamp | 700 |
| Flexing Region | 740 |
| First Clamp Handle | 710 |
| Second Clamp Handle | 720 |
| Flexing Part | 730 |
| Coiled Spring | 735 |
| Coiled Region | 735a |
| Longitudinal End | 735b |
| Longitudinal End | 735c |
| Locking hub | 800 |
| Locking Neck Region | 820 |
| Locking Tip Region | 840 |
| First Locking Tip | 841 |
| Second Locking Tip | 842 |
| Locking Protrusion | 843 |
| Locking Recess | 844 |
| Locking Recess | 845 |
| Locking Protrusion | 846 |
| Splittable Needle Assembly | 1000 |

### III. Kits

Kits containing plurality of individually wrapped and sterilized splittable needles and instructions for use are also provided. Optionally, the kits may include sterilized and packaged guidewires and PICC and/or Midline catheters. In preferred embodiments, the kits contain a plurality of individually wrapped and sterilized splittable needles of various sizes.

### IV. Methods of Use

### A. Assembly

The splittable needles described herein are suitable for use in cardiac catheterization through peripherally located blood vessels. The splittable needles are assembled by aligning and simultaneously locking the two or more parts of the splittable needles.

The aligning and locking is achieved by aligning the one or more protrusions of one needle part with one or more notches of the other needle part and pressing on the parts to insert the protrusion(s) into the notch(es). Once inserted, the needle parts are assembled into a splittable needle, with the parts precisely aligned for ease of insertion into tissue and for minimizing leakages from areas of contact.

### B. Threading

The splittable needle assembly is then inserted into a body lumen of a subject in need thereof. The body lumen is typically any vein or artery.

The splittable needle is inserted so that the rear end remains outside of the body and accessible by clinicians. A guidewire is then threaded through the needle. The guidewire is first entered into the funnel region in the rear end of the needle, then threaded through the needle and through the beveled tip into the vessel.

### C. Retracting

After the guidewire is placed inside the vessel, the splittable needle assembly is retracted. Following retraction, the tip at the front end of the needle is no longer inside the vessel, but the needle is still threaded through the guidewire. The splittable needle is ready to be split and removed from the guidewire.

### D. Splitting

The splittable needle is split by pressing on the radially protruding clamp handles of the flexing region to bring the clamp handles closer together. This motion allows the clamp handles to come towards while simultaneously pulling apart the securing regions. As the securing regions are pulled apart, they in turn pull away the protrusion(s) from the notch(es). This motion therefore separates one longitudinal needle part from the other, splitting the needle into individual parts that it can be removed from the guidewire.

### E. Applications

1. Cardiac Catheterization

During vascular access for cardiac catheterization, the first step is to use an “introducer” needle to access the blood vessel. The next step is to introduce the guidewire, followed by catheter insertion. The introducer needle is removed after the guidewire is inserted. In most of the cardiac catheterization procedures, the guidewires are around six feet long, and the needle has to be retracted along the entire length of the guidewire before continuing the catheterization procedure. The needle described herein avoids this retraction step by “opening” along its length, so that the needle can be removed immediately after the guidewire is inserted into the blood vessel thereby improving the efficiency and safety of the procedure.

2. Percutaneous Coronary Intervention

Angioplasty, also called percutaneous coronary intervention (PCI), is a procedure used to open blocked coronary arteries (caused by coronary artery disease) and restore blood flow to the heart muscle without open-heart surgery.

For angioplasty, a special catheter (a long, thin, hollow tube) is inserted into a blood vessel and guided to the blocked coronary artery over a guidewire which is either 190 or 300 cm long. The guidewire has a very sensitive tip which is either custom-shaped or pre-shaped. It is introduced into the catheter via a blunt needle introducer. The catheter has a tiny balloon at its tip. Once the catheter is in place, the balloon is inflated at the narrowed area of the coronary artery. This presses the fatty tissue against the sides of the artery making more room for blood flow.

The splittable needles described herein can aid in insertion of the catheter into the blood vessel during PCI by allowing the blunt needle to be removed from the wire without needing to pull through its whole length by splitting.

3. Biopsy

The splittable needles described herein can have applications in diverse biopsy procedures. Biopsies accessing hard-to-reach tissues require inserting into patient's body and guiding long flexible wire-like instruments, such as laparoscopes and bioptomes, to a desired tissue site using x-ray or ultrasound guidance. Splittable needles may aid in inserting of the instruments at a particular location in the body, and then can be removed by retracting the needle from the body and splitting it into two parts to separate the needle from the instrument.

For example, endomyocardial biopsies require insertion and guidance of bioptomes from blood vessels to the heart for sampling of cardiac tissue following cardiac transplantation (Kilo et al., Multimedia Manual of Cardiothoratic Surgery, doi:10.1510/mmcts.2005.001149 (2006)). The splittable needles could be used for placement of the bioptomes in the desired blood vessel.

Liver biopsies require insertion into tissue and guidance of long needles to the liver. The splittable needles can be used to insert and position the biopsy needle, but then be retracted and removed from the biopsy needle following collection of a liver sample.

Splittable needles can also be useful for guiding needle biopsies into soft tissues e.g. breast. The splittable needles can precisely guide the biopsy needles into tissues of interest and then be retracted and split away from the biopsy needle.

### V. Examples

### Example 1. Use of the Splittable Needle for Catheterization of a Laboratory Animal

Using ultrasonographic guidance, vascular access was obtained in a pig's femoral artery using the splittable needle. A 180 cm 0.035-inch diameter guidewire was placed in the common femoral artery and the needle was successfully removed over the wire using the splittable technique instead of withdrawing over the whole wire.

We claim:

1. A splittable needle assembly comprising:
a splittable needle comprising a hollow shaft having opposed distal ends and proximal ends, the distal end having a tip for insertion into a body and the proximal end having a locking hub, wherein the hollow shaft comprises at least two elements joined together to form the hollow shaft of the splittable needle; and
a clamp holding the at least two elements together to form the splittable needle, wherein the clamp is positioned at the proximal end of the hollow shaft before the locking hub and wherein the clamp comprises an alignment element for aligning the at least two elements and tightly joining together to form the hollow shaft of the splittable needle.

2. The splittable needle assembly of claim 1, wherein the clamp comprises at least two handles and a securing region, wherein the at least two handles are radially protruding from the securing region, and are flexibly joined.

3. The splittable needle assembly of claim 2, wherein the alignment element is located at the securing region of the clamp.

4. The splittable needle assembly of claim 1, wherein the at least two elements are magnetically joined together to form the hollow shaft of the splittable needle.

5. The splittable needle assembly of claim 1, wherein the at least two elements are coated by a polymer-mixture coating and joined together to form the hollow shaft of the splittable needle.

6. The splittable needle assembly of claim 1, wherein the at least two elements are joined together with a biodegradable adhesive to form the hollow shaft of the splittable needle.

7. The splittable needle assembly of claim 1, wherein the alignment element is formed of matingly-fitted elements comprising protrusions, recessions, cuts, and stepped ridges.

8. The splittable needle assembly of claim 1, wherein the alignment element comprises a protrusion on a first arm of the clamp that matingly engages a recession in a second arm of the clamp to align the at least two elements when the first and second arms of the clamp close to form the hollow shaft of the splittable needle.

9. The splittable needle assembly of claim 1, wherein the clamp is a spring-loaded clamp.

10. The splittable needle assembly of claim 2, wherein the at least two elements separate when the clamp handles are brought closer together.

11. The splittable needle assembly of claim 1, wherein the at least two elements separate when introduced into a body lumen.

12. The splittable needle assembly of claim 1, wherein the needle in its assembled form has a size ranging from 7 Gauge (g) to 33 g.

13. The splittable needle assembly of claim 1, wherein the locking hub is configured for locking with additional devices comprising syringes, delivery devices, valves, filters, pumps and pouches.

14. The splittable needle assembly of claim 1, wherein the locking hub comprises a locking alignment element.

15. The splittable needle assembly of claim 14, wherein the locking hub comprises a locking alignment element formed of matingly-fitted elements comprising protrusions, recessions, cuts, and stepped ridges.

16. A kit comprising a plurality of individually wrapped and sterilized splittable needle assemblies and instructions for use.

17. A method of accessing a body lumen or tissue of a subject comprising inserting into the subject the splittable needle assembly of claim 1.
